(19) United States
(12) Patent Application Publication
(10) Pub. No.: US 2021/0121642 A1
(43) Pub. Date: Apr. 29, 2021

(54) PEN NEEDLE ASSEMBLY

(71) Applicant: Becton, Dickinson and Company, Franklin Lakes, NJ (US)

(72) Inventors: Victor POLITIS, Natick, MA (US); J. Richard GYORY, Sudbury, MA (US); Joseph ISKANDAR, Allston, MA (US); Alex CHAVES, Tyngsboro, MA (US)

(73) Assignee: Becton, Dickinson and Company, Franklin Lakes, NJ (US)

(21) Appl. No.: 17/051,716

(22) PCT Filed: Apr. 26, 2019
(86) PCT No.: PCT/US19/29272

§ 371 Date: Oct. 29, 2020

Related U.S. Application Data
(60) Provisional application No. 62/664,418, filed on Apr. 30, 2018.

(51) Int. Cl. A61M 5/34; A61M 5/32

(52) U.S. Cl. CPC A61M 5/345; A61M 2207/00; A61M 5/3293

(57) ABSTRACT

A pen needle assembly includes an adapter (32, 122, 190, 254, 312) and a needle hub (134, 124, 192, 256, 314) coupled to the adapter. The needle hub is removable from the adapter. The adapter has a distal end for coupling with a pen needle delivery device and a distal end with at least one coupling member for coupling with the needle hub. The needle hub is a distal face forming a skin contact surface and a cannula extending from the distal face. The proximal end of the needle hub includes a coupling member for removably coupling with the coupling member of the adapter.

This application claims priority to U.S. Provisional Patent Application No. 62/664,418, filed on Apr. 30, 2018, which is hereby incorporated by reference in its entirety.

### FIELD OF THE INVENTION

The present invention relates generally to a pen needle assembly for use with a delivery device, such as a pen delivery device. The pen needle assembly in one embodiment includes an adapter for coupling with a needle hub for use with a pen needle delivery device.

### BACKGROUND OF THE INVENTION

Insulin and other injectable medications are commonly delivered with drug delivery pens, whereby a disposable pen needle hub is attached to the pen to facilitate access to a drug container and allow fluid egress from the container through the needle into the patient.

Various pen needle delivery devices are known in the art for dispensing the substance to the patient. The delivery devices often use a disposable needle hub having a cannula or needle extending from a patient end of the hub for inserting into the patient. A non-patient end of the hub is coupled to the pen delivery device for delivering the substance to the patient.

The pen needle is often packaged in a container containing several loose needle hub assemblies. A needle hub is removed from the package and attached to the pen needle delivery device for injecting the patient and then removed from the delivery device to be discarded. The needle hub assembly includes an outer cover that encloses the needle hub and a removable seal that is peeled from the outer cover to open the cavity so that the needle hub can be removed. The needle hub can have threaded non-patient end that is threaded onto the delivery device. The delivery device with the attached needle hub is then removed from the outer cover. An inner needle shield is attached to the needle hub to cover the cannula until the device is ready for use. The inner shield is removed to expose the cannula for use to deliver the substance to the patient. After use, the needle hub can be inserted back into the outer cover to enclose the exposed cannula. The pen delivery device is separated from the needle hub leaving the needle hub within the outer cover.

The prior devices require the removing and replacing the pen needle between uses by separating the pen needle from the pen needle delivery device. Separation of the pen needle normally requires unscrewing the used pen needle and screw an new pen needle onto the pen needle delivery device.

Existing pen needle assemblies are disclosed in U.S. Patent Application Publication Nos. 2006/0229562 to Marsh et al. and 2007/0149924 to R. Marsh, the entire contents of both of which are hereby incorporated by reference for this purpose.

Although the prior devices have been suitable for the intended use, there is a continuing need in the industry for improved packaging for a pen needle hub assembly.

### SUMMARY OF THE INVENTION

The present invention is directed to a pen needle assembly having a pen needle that can be attached to and removed easily from an injection apparatus and to a method of use of the pen needle assembly.

The pen needle assembly includes an adapter for coupling with the outlet end of the pen needle delivery device and a needle hub that can be coupled to the end of the adapter. The adapter in one embodiment has a threaded coupling for connecting to a threaded end of the pen needle delivery device. The needle hub has a needle projecting from a distal face and a projection extending from a proximal end for coupling to the distal end of the adapter. The needle hub is disposable and replaced after use with a new needle hub without the need to remove the adapter from the pen needle delivery device.

The needle hub has a shape and dimension for supporting a needle for injecting the substance, such as insulin, into the patient. The needle hub has a distal face forming a skin contact surface surrounding the needle to contact the surface of the skin during use and limiting and controlling the depth of penetration of the needle into the skin. In one embodiment, the contact surface has a substantially convex surface or profile with a curvature to control the deformation of the surface of the skin and control the depth of penetration.

The needle hub has a proximal end with a shape and configuration for coupling with the adapter and forming a fluid connection between the delivery device and the needle. In one embodiment, the adapter includes a septum that is pierced by a projection extending from the needle hub when coupled to the adapter. The projection has a fluid passage forming the fluid connection between the delivery device and the needle.

The needle hub has a coupling mechanism for mating with a complementing coupling mechanism on the adapter. The coupling mechanism can be one or more hooks, tabs, or threads that connect with complementing hooks, tabs, threads or other connecting structure on the adapter. In one embodiment, the adapter includes the plurality of legs extending from the distal end of the adapter. Each of the legs has a hook that connects to the complementing hook or recess on the needle hub. The hooks on the legs can project radially inward for connecting with the flange on the needle hub. The surfaces of the hooks and a flange can be angled or chamfered to assist in assembling the needle hub onto the adapter. In other embodiments, the hooks on the adapter can project outwardly for connecting with complementing hooks on the needle hub.

In other embodiments, the adapter can be connected to the needle hub by a friction fit or an interference fit. The needle hub in one embodiment includes a collar that extends from the proximal end of the needle hub for mating with a complementing surface on the adapter. The collar on the adapter can have an inner surface with a detent projecting radially inward for coupling with the complementing recess on an outer surface of the adapter. In other embodiments, the opposing surfaces of the adapter and needle hub are substantially smooth and have a dimension to form a friction fit or press fit.

In a further embodiment, the needle hub can have a sleeve with internal threads for coupling with external threads on the adapter. The threads are configured to provide a quick connect and disconnect while providing a fluid tight seal with the adapter. The threads on the needle hub can have a pitch greater than the axial length of the needle hub so that the needle hub can be attached and disconnected by rotating the needle hub less than one full rotation relative to the adapter.

The pen needle in another embodiment includes a body with a distal end with a distal surface forming a skin contact surface during use and a proximal end for connecting to the pen needle delivery device. The proximal end of the body has a collar extending axially from the distal end of the body. The collar includes internal threads for connecting to the pen needle delivery device. The collar is formed by a plurality of flexible legs that can deflect when connected to the pen needle delivery device. The legs are formed with the concave inner surface with the threads formed on the concave inner surface. In one embodiment, the legs can be spaced apart to define a gap between the adjacent legs and having an axial length extending from the distal end of the collar to the proximal end of the collar.

In other embodiments, the collar can have a continuous proximal end and a distal end connected to the body. Cutouts are formed in the sidewalls of the collar forming the flexible tabs where the flexible tabs are connected to the proximal end of the collar and have a free end oriented toward the distal end of the collar. In further embodiments, the collar can be formed with flexible tabs projecting in a distal direction with respect to the collar and tabs extending in a proximal direction with respect to the collar.

The pen needle assembly basically comprises an adapter and a needle hub. The adapter has a sidewall with an open proximal end configured for coupling to a pen needle delivery device and a distal end with an end wall. The end wall has a fluid pathway for connecting with the delivery device. The needle hub has a distal end with a distal face, a needle extending from the distal face for delivering a substance from the pen needle delivery device, and a proximal end. The proximal end is coupled to the adapter by a suitable coupling mechanism and is removable from the adapter.

The pen needle assembly comprises a body and a side wall in one embodiment. The body has a distal end, a proximal end and an end wall with an axial face and the needle extending from the end wall. The side wall extends from the proximal end of the body where the side wall has a substantially cylindrical configuration. The side wall has an outer face and an inner face with internal threads for coupling to a threaded end of the delivery device. The side wall has a plurality of flexible legs for coupling with the delivery device.

The objects, advantages, and salient features of the invention will become apparent from the following detailed description, which, taken in conjunction with the annexed drawings, discloses exemplary embodiments of the invention.

### BRIEF DESCRIPTION OF THE DRAWINGS

The above benefits and other advantages of the various embodiments of the present invention will be more apparent from the following detailed description of exemplary embodiments of the present invention and from the accompanying figures, in which:

FIG. 1 is an exploded perspective view of a pen needle delivery device in one embodiment showing the pen needle assembly that includes a needle hub supporting a cannula, inner shield, and outer cover;

FIG. 2 is a perspective view of the pen needle assembly in one embodiment;

FIG. 3 is a perspective view of the needle hub in the embodiment of FIG. 2;

FIG. 4 is a cross-sectional view of the pen needle assembly of FIG. 2;

FIG. 5 is a perspective view of the adapter in the embodiment of FIG. 2;

FIG. 6 is a perspective view of the pen needle assembly in another embodiment;

FIG. 7 is a cross-sectional view of the pen needle assembly of the embodiment of FIG. 6;

FIG. 8 is a perspective view of the adapter of the embodiment of FIG. 6;

FIG. 9 is a cross-sectional of the adapter showing the septum;

FIG. 10 is a top perspective view of the needle hub;

FIG. 11 is a bottom perspective view of the needle hub;

FIG. 12 is a perspective view of the pen needle assembly in another embodiment;

FIG. 13 is a cross-sectional view of the adapter of the embodiment of FIG. 12;

FIG. 14 is a cross-sectional view of the pen needle assembly of FIG. 12;

FIG. 15 is a perspective view of adapter;

FIG. 16 is a perspective view of the needle hub;

FIG. 17 is a cross-sectional view of the needle hub;

FIG. 18 is a perspective view of a further embodiment;

FIG. 19 is a partial cross-sectional view showing the needle hub and adapter of FIG. 18;

FIG. 20 is a perspective view of the needle hub;

FIG. 21 is a perspective view of the adapter;

FIG. 22 is a perspective view of the pen needle assembly in a further embodiment;

FIG. 23 is a partial cross-sectional view showing the needle hub and adapter of FIG. 22;

FIG. 24 is a cross-sectional view of the needle hub;

FIG. 25 is a perspective view of the adapter;

FIG. 26 is a cross-sectional view of the adapter;

FIG. 27 is a perspective view of the needle assembly in another embodiment;

FIG. 28 is a side view showing the needle hub attached to the adapter with the connecting threads shown in phantom lines of the embodiment of FIG. 27;

FIG. 29 is a bottom perspective view of the needle hub showing the connecting threads;

FIG. 30 is a perspective view of the adapter showing the connecting threads;

FIG. 31 is a perspective view of the needle hub assembly having a needle hub with projecting legs forming a side wall in another embodiment;

FIG. 32 is a side view of the assembly of FIG. 31;

FIG. 33 is a perspective view of the needle hub assembly in another embodiment;

FIG. 34 is a side view of the assembly of FIG. 33;

FIG. 35 is a perspective view of the needle hub assembly in another embodiment;

FIG. 36 is a side view of the assembly of FIG. 35;

FIG. 37 is a perspective view of the needle hub assembly in another embodiment;

FIG. 38 is a side view of the assembly of FIG. 37;

FIG. 39 is a cross-sectional view of the septum in one embodiment; and

FIG. 40 is a cross-sectional view of the septum in another embodiment.

Throughout the drawings, like reference numbers will be understood to refer to like parts, components, and structures.

### DETAILED DESCRIPTION OF THE EXEMPLARY EMBODIMENTS

The present invention is directed to a pen needle assembly apparatus for use with an injection delivery device, such as a pen needle delivery. The invention is further directed to a pen needle assembly having an adapter that is connected to the pen needle delivery device and a needle hub that can be coupled to the adapter. The adapter is configured to provide fluid communication from the pen needle delivery device to the needle hub.

Reference will now be made in detail to embodiments of the present invention, which are illustrated in the accompanying drawings, wherein like reference numerals refer to like elements throughout. The embodiments described herein exemplify, but do not limit, the present invention by referring to the drawings. The exemplary embodiments are presented in separate descriptions, although the individual features and construction of these embodiments can be combined in any number of ways to meet the therapeutic needs of the user.

This disclosure is not limited in its application to the specific details of construction and the arrangement of components set forth in the following description or illustrated in the drawings. The embodiments herein are capable of being modified, practiced or carried out in various ways. Also, it will be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not limited to physical or mechanical connections or couplings. Further, terms such as up, down, bottom, and top are relative, and are to aid illustration, but are not limiting. The features of the embodiments described herein can be combined with features of other embodiments as long as the features are not inconsistent with one another. The term “substantially” is intended to mean mostly but not necessarily entirely. Terms of degree, such as “approximately” or “substantially” are understood by those skilled in the art to refer to a reasonable ranges around and including a given range or value, for example, general tolerances associated with manufacturing, assembly and the use of the described embodiments.

The apparatus for the needle hub assembly enables the convenient and easy assembly and removal of the needle hub on the pen delivery device. The needle hub is easily attached to and removed from the adapter that is coupled to the pen delivery device.

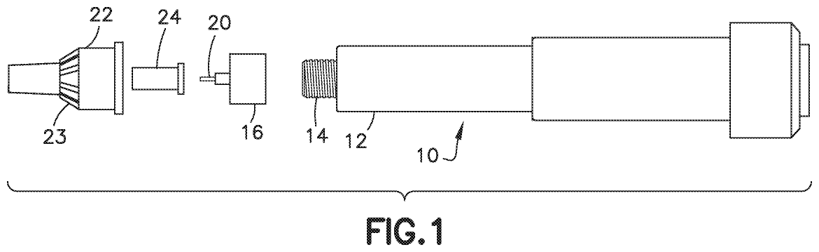

Delivery device 10, as shown in FIG. 1 typically comprises a dose knob/button, an outer sleeve 12, a threaded end 14, and a cap. A dose knob/button allows a user to set the dosage of medication to be injected. The outer sleeve 12 is gripped by the user when injecting medication. The cap is used by the user to securely hold the pen injector device 10 in a shirt pocket or other suitable location and provide cover/protection from accidental needle injury.

In standard pen needle delivery devices the dosing and delivery mechanisms are all found within the outer sleeve 12 and is not described in greater detail here as they are understood by those knowledgeable of the prior art. A medicament cartridge is typically attached to a standard pen injector housing by known attachment means. The distal movement of a plunger or stopper within the medicament cartridge causes medication to be forced into the reservoir housing. The medicament cartridge is sealed by a septum and punctured by a septum penetrating needle cannula located within a reservoir or housing. Reservoir housing is preferably screwed onto the medicament cartridge although other attachment means can be used. The pen needle delivery device can be a standard pen delivery device known in the industry so that the pen needle delivery device is not shown in detail. The pen needle assembly 18 as shown in FIG. 1 includes a needle hub 16 supporting a cannula 20, an outer cover 22, and an inner shield 24. A protective seal is attached to the open end of the outer cover to enclose the needle hub and cannula to maintain a clean and sterile condition. The seal is easily peeled from the outer cover to access the needle hub during use.

The delivery device 10 is connected to needle hub 16 shown in FIG. 1 that has a connecting non-patient end with internal threads that screw onto the threaded end 14 of the delivery device 10. The needle cannula 20 extends from the patient end of the needle hub 16 for delivering the substance to the patient. The outer cover 22 covers the needle cannula to protect the patient from accidental needle stick before and after use. The outer cover 22 includes ribs 23 to assist in gripping the outer cover during use. The inner shield 24 is provided over a post extending from the end of the needle hub 16 to enclose the cannula. During use, the needle hub 16 is connected to the pen delivery device and the inner shield is removed. After use, the outer cover is generally placed back on needle hub to cover the needle cannula. The needle hub with the cover is then removed from the pen needle delivery device and discarded.

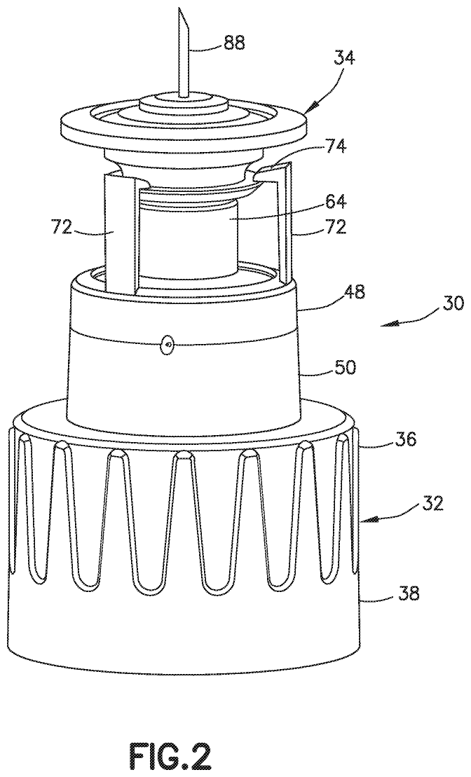

Referring to FIGS. 2-5, a first embodiment of the pen needle assembly 30 is configured for coupling to the pen needle delivery device 10 in a manner substantially as shown in FIG. 1. The pen needle assembly 30 includes an adapter 32 and a needle hub 34 for coupling the adapter 32. The adapter 32 and the needle hub 34 are typically made of a rigid plastic material. In the present description, the delivery device refers to a pen needle delivery device substantially as shown in FIG. 1 so that the terms can be used interchangeably. The delivery device can be the device 10 of FIG. 1 or other suitable device that is able to provide controlled dosage of the substance to the patient.

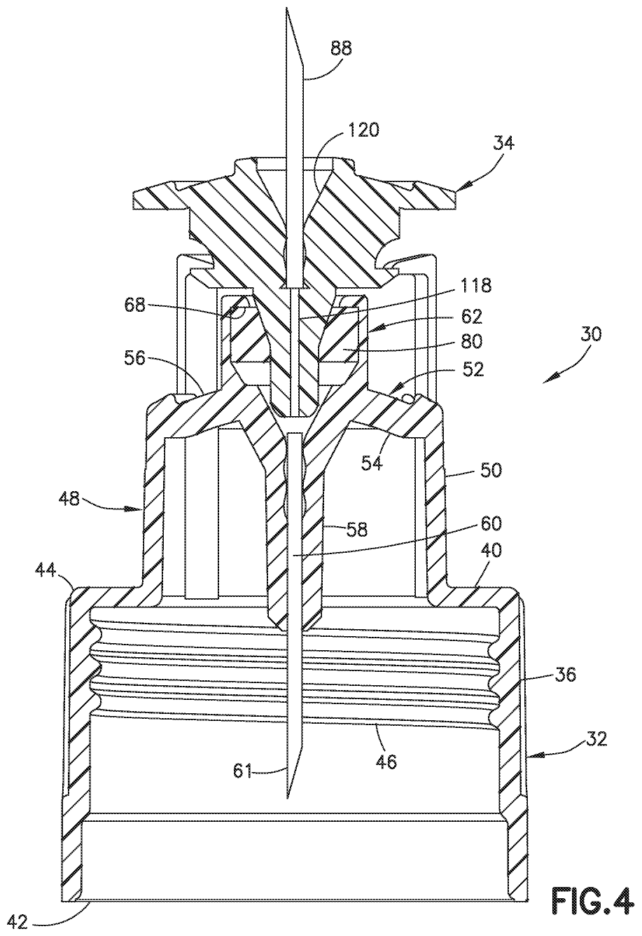

Adapter 32 has a proximal end with a shape and configuration similar to the pen needle assembly of FIG. 1 for connecting to the dispensing end of the pen needle delivery device by a thread connection. The adapter 32 in the embodiment shown has a body 36 defined by a side wall 38 and a top wall 40. Side wall 38 includes an open proximal end 42 for coupling with the delivery device and a distal end 44 formed with the top wall 40. The side wall 38 includes internal threads 46 for coupling to a threaded end of the delivery device as shown in FIG. 4.

An end portion 48 extends from the top wall 40 and is formed by a sidewall 50. An end wall 52 closes the distal end of the end portion 48 of the body 36 and sidewall 50 as shown in FIG. 4. In the embodiment shown, the end wall 52 has an inner face 54 and an outer face 56. A post 58 extends from the inner face 54 toward the proximal end 42. The post 58 has an axial passage 60 shown in FIG. 4. A cannula or needle 61 can be received within the axial passage 60 that is configured for piercing a septum in the delivery device for making a fluid connection between the delivery device and the adapter 32. The cannula 61 extends into the cavity formed by the side wall 38 and is typically spaced inwardly from the proximal end of the side wall.

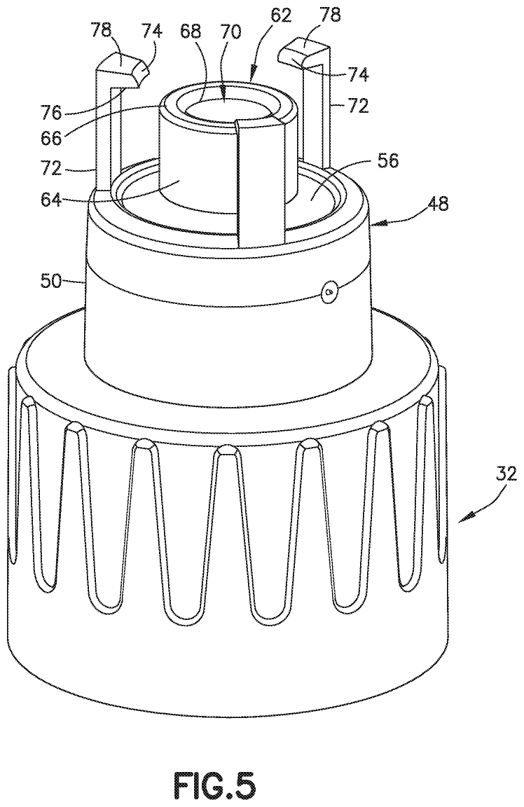

The outer face 56 of the end wall 52 of end portion 48 forms a distal face of the adapter. In the embodiment shown, the outer face has a generally convex configuration although other configurations can be used. A collar 62 extends axially from the outer face 56 in a distal direction. The collar 62 in the embodiment shown has a substantially cylindrical sidewall with a distal end 66, and a flange 68 extending radially inward and defining a distal opening 70 as shown in FIGS. 4 and 5. The inwardly extending flange 68 in the embodiment shown has a beveled or chamfered distal face as shown in FIG. 4.

A coupling mechanism is provided on the adapter 32 for coupling with the needle hub 34. In the embodiment shown in FIG. 5, the coupling mechanism is provided on the outer face 56 and includes at least one, and typically a plurality of legs 72 extending from the outer face 56 in a distal direction. In the embodiment of FIGS. 2-5 three legs 72 are spaced around the circumference of the outer face 56. Each leg 72 can have a distal end with an inwardly extending tab 74 for coupling with the needle hub 34. Each tab 74 has a substantially flat inner face 76 extending in a plane perpendicular to the longitudinal axis and an inclined or chamfered outer distal face 78.

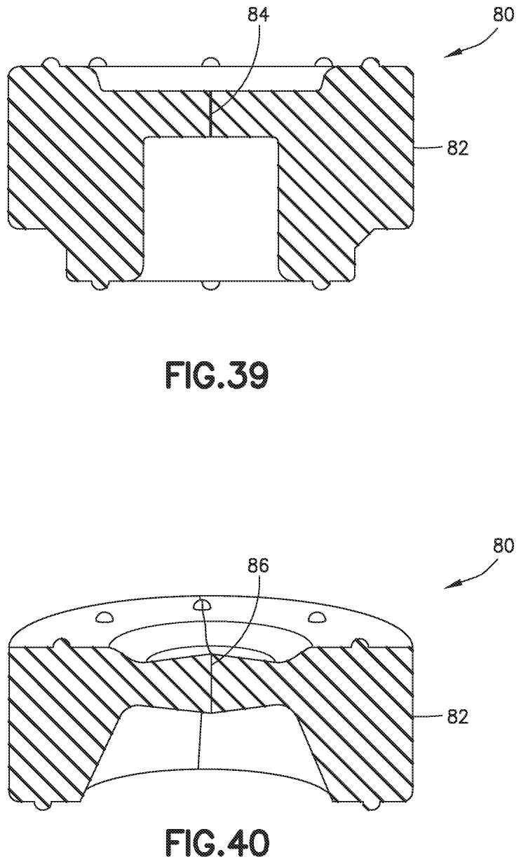

Collar 62 has an inner cavity for receiving a septum 80 as shown in FIG. 4. The septum 80 as shown in FIG. 4 is positioned in the cavity of the collar 62 for forming a fluid tight seal between the adapter 32 and the needle hub 34. The septum 80 can have various configurations that are able to form a seal to prevent fluid leakage while enabling the needle hub 34 to penetrate the septum. One example of a septum is shown in FIG. 39. The septum 80 has a substantially cylindrical configuration with an outer surface 82 for forming a friction fit with the inner surface of the collar 62. The septum 80 includes a slit 84 that can be penetrated by the proximal end of the needle hub. The septum is typically made of a flexible polymeric material that is able to form a fluid seal while allowing the needle hub to pass through the slit 84. FIG. 40 shows a similar embodiment of a suitable septum having a slit 86 for allowing penetration by the needle hub 34.

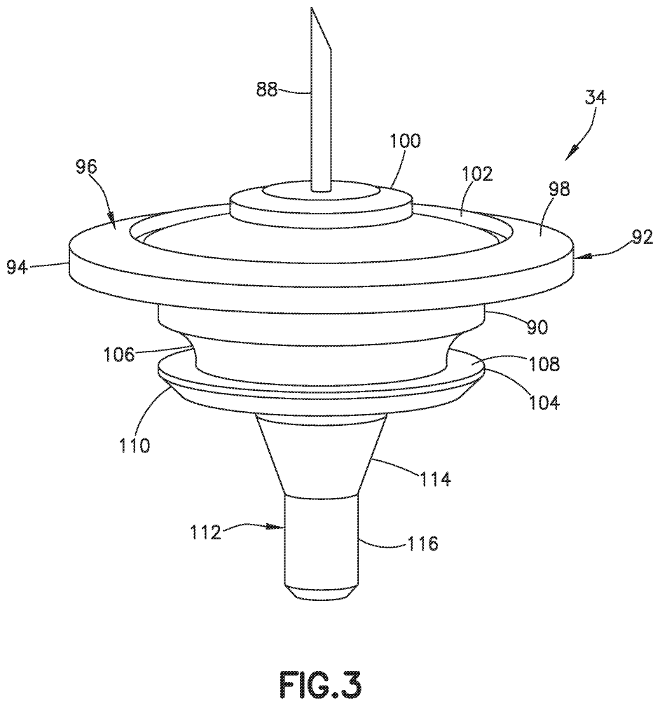

Needle hub 34 shown in FIG. 3 has a shape and configuration for supporting a needle 88 or cannula for injecting the substance from the delivery device into the patient. The needle 88 has a suitable gauge and length for penetrating the skin to a selected depth for delivery of the substance to a controlled depth in the skin. The needle 88 includes a lumen for delivering the substance to the patient. The needle hub 34 in the embodiment shown in FIG. 3 has a body 90 with a substantially circular shape. The body 90 has a proximal end and a distal end. The distal end of the body 90 has a skin contact member 92 forming a distal face of the needle hub 34. The contact member 92 has a substantially circular configuration with a diameter greater than the diameter of the body 90. In the embodiment shown, contact member 92 has a cylindrical side surface 94 and a distal surface 96 forming a skin contact surface.

The distal surface 96 has an outer annular ring shaped skin contact surface 98 and an inner annular ring shaped skin contact surface 100. An annular recess 102 is formed between the outer annular surface 98 and the inner annular surface 100. In the embodiment shown, the distal surface 96 has a substantially convex configuration so that the inner annular surface 100 is spaced outwardly relative to the outer annular surface 98 in the distal axial direction. The distal surface 96 has a configuration for contacting the skin during the injection and deforming a skin in a controlled manner to control the depth of penetration of the needle 88. The distal skin contact surface 96 has a substantially convex shape.

The proximal end of the body 90 includes an annular flange 104 extending radially outward and forming an annular recess 106 between the annular flange 104 and the body 90. The annular recess 106 has a dimension for complementing the tabs 74 of the needle hub for coupling the needle hub 34 to the adapter 32. The flange 104 in the embodiment shown has a substantially flat distal face 108 and an inclined proximal face 110 for sliding over the tabs 74. A projection 112 extends from the proximal end of the body 90 as shown in FIG. 3 and FIG. 4. The projection 112 has an upper conical portion 114 adjacent the body 40 and a substantially cylindrical end portion 116 at a proximal end of the needle hub 34. A fluid passage 118 shown in FIG. 4 extends axially through the needle hub 34 between the proximal end of the projection 112 and the distal face 90. As shown in FIG. 4 the passage 118 terminates at the distal end forming a conical shaped recess 120 for receiving an adhesive to bond the needle 88 to the needle hub 34.

The adapter 32 is connected to the threaded end of the delivery device in a manner similar to the device shown in FIG. 1. The post 58 and the cannula 61 extending from the post of the adapter are able to penetrate a septum in the delivery device to form the fluid connection between the delivery device and the septum 80. Once the adapter 32 is connected to the delivery device, the needle hub 34 is connected to the adapter 32.

In the embodiment shown, the needle hub 34 snaps onto the coupling mechanism of the adapter by inserting the projection 112 through the opening 70 in the collar 62 where the projection 112 passes through the septum 80. A downward linear force presses the flange 68 over the ends of the legs 72 so that the legs deflect outwardly by the complementing inclined surface 78 on the legs and the inclined surface 110 on the flange. The tabs 74 on the respective leg 72 snap into the annular recess 106 to couple the needle hub 34 to the adapter 32. The legs 72 are sufficiently flexible and resilient to bend outwardly and slide over the flange 104 to couple to the needle hub 34. In the assembled position shown in FIG. 4, the passage 118 forms a fluid connection with the passage 60. The delivery device injects and delivers the substance to the patient. After use, the used needle hub is removed by an outward force to disconnect the legs 72 from the flange 104. The needle hub can be pulled axially to separate from the adapter. The septum 80 closes after removal of the used needle hub 34 to prevent leakage from the delivery device and the adapter. A new needle hub can then be connected to the adapter for delivering a subsequent dosage of the substance to the patient.

The needle hub 34 can be packaged with a suitable outer cover and seal to maintain the needle hub in a sterile condition. The outer cover can be similar the embodiment of FIG. 1 where the needle hub is retained in the outer cover by a friction fit. The pen needle can then be snapped onto the adapter and the outer cover removed to expose the needle without the need for the user to handle the pen needle with the needle exposed.

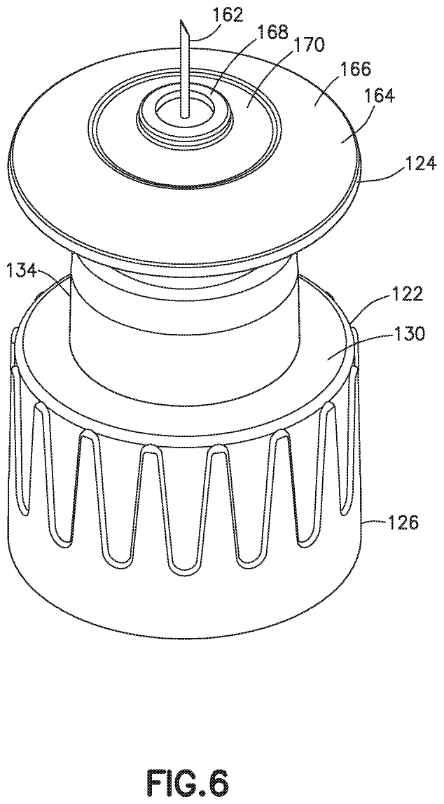

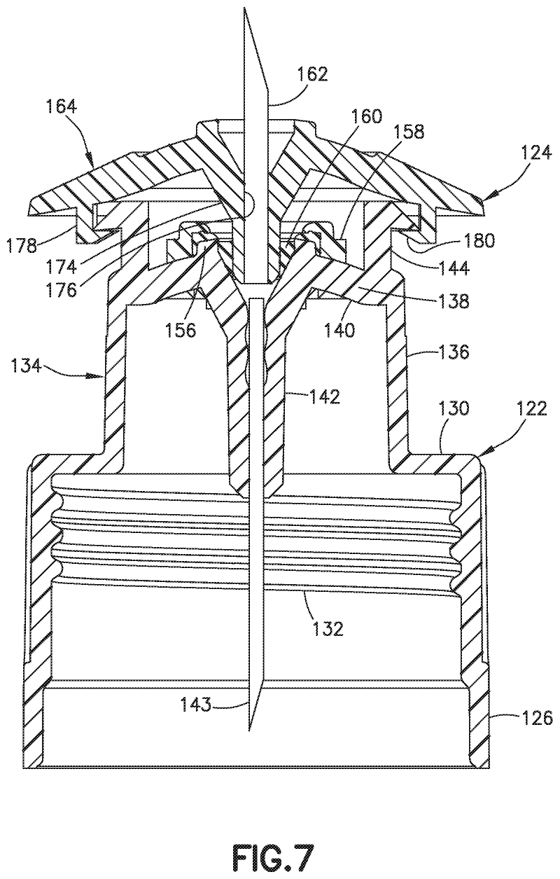

Another embodiment of the pen needle assembly as shown in FIGS. 6-11 includes an adapter 122 and a needle hub 124. Needle hub 124 is similar to the needle hub in the previous embodiment having a body with a sidewall 126 forming an open proximal end 128 and having top wall 130. Internal threads 132 provide a connection to the delivery device. A cylindrical end portion 134 having a cylindrical side wall 136 extends distally from the top wall 130 to an end wall 138. The end wall 138 has an inner surface 140 with a post 142 extending toward the proximal end of the adapter as shown in FIG. 7. The post 142 has an axial passage that receives a cannula 143 for piercing the septum of the delivery device.

In the embodiment shown, the adapter 122 has a coupling mechanism for coupling with the needle 124. The coupling mechanism is formed by an annular wall 144 extending axially from the distal end of the adapter and has a distal end with an outwardly projecting lip 146. The lip 146 in the embodiment shown has a substantially flat bottom surface 148 and an inclined top surface 150. As shown in FIG. 7, the annular wall 144 extends axially from the end wall 138. In the embodiment shown in FIG. 7, the annular wall 144 is oriented at or close the peripheral edge of the end wall 138.

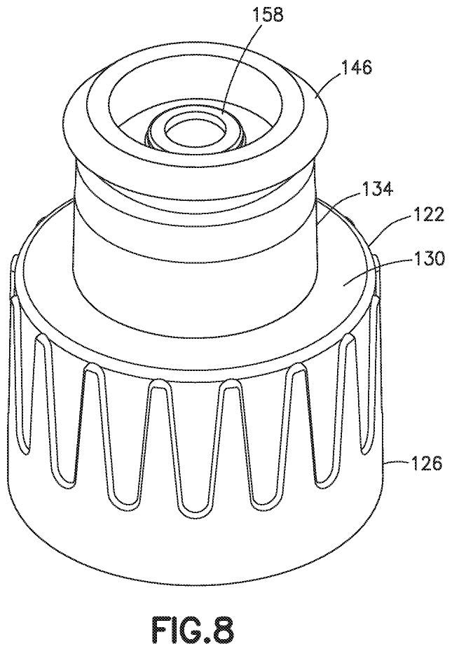

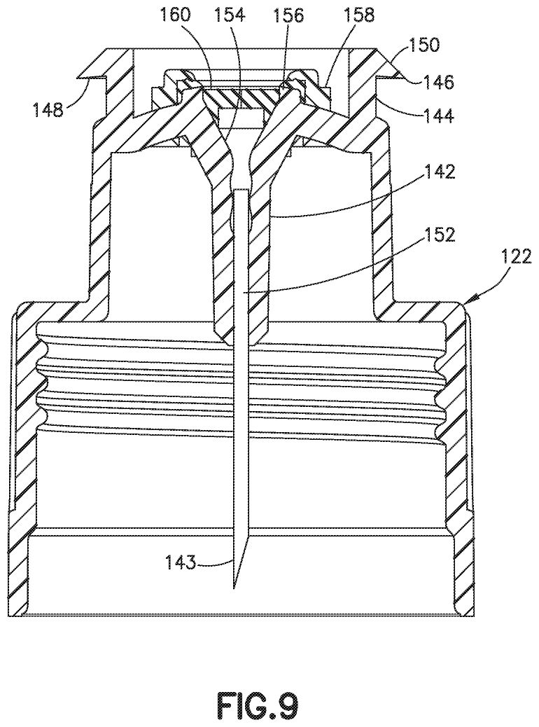

The post 142 has an axial passage 152 shown in FIG. 9 that terminates at a conical shaped recess 154 at the distal end of the adapter 122 at the end wall 138. An annular ridge 156 surrounds the conical shaped recess 154 and projects axially outward. An outer annular ridge 158 surrounds the inner annular ridge 156 for supporting a septum 160 as shown in FIG. 7. As in the previous embodiment, the septum is made of a flexible polymeric material and has a slit that can be penetrated by the needle hub 124.

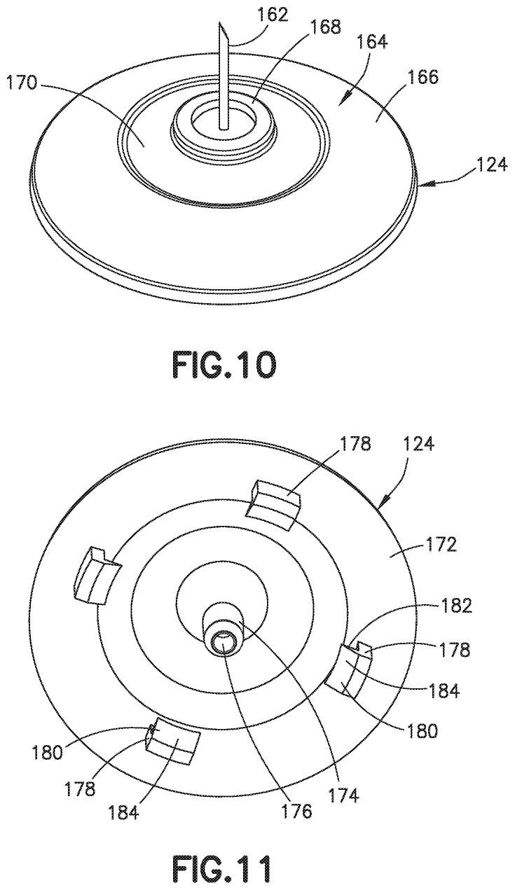

The needle hub 124 supports a needle 162 for delivering the substance to the patient. The needle hub 124 as shown in FIG. 10 has a distal face 164 forming a skin contact surface during penetration of the needle. The distal face 164 in the embodiment shown has an outer annular surface 166 and an inner annular surface 168 forming an annular recess 170 between the convex outer annular surface 166 and the convex inner annular surface 168. The annular recess 170 has a bottom wall with a radius of curvature that is substantially the same as the radius of curvature of the inner ring 168 and the outer ring 166. As in the previous embodiment, the distal face 164 has a substantially concave configuration. As shown in FIG. 10, the outer annular surface 166 has a radial width greater than the radial width of the inner annular surface as measured between the outer peripheral edge and the inner edge. In one embodiment, the outer annular surface 166 has a radial width of about 3 to 5 times the radial width of the inner annular surface 168.

The needle hub 124 has a proximal face 172 configured for coupling with the adapter 122. A projection 174 shown in FIG. 7 extends from the proximal face. The projection 174 includes an axial passage 176 for supporting the needle 162 and providing fluid communication between the delivery device and the needle. The projection 174 has an axial length sufficient to pierce and penetrate the septum 160 of the adapter as shown in FIG. 7.

The needle hub 124 includes a coupling mechanism defined by one or more connecting legs 178 extending from the proximal face 172. As shown in FIG. 11, the legs are uniformly spaced around the center axis of the needle hub and the projection 174. In the embodiment shown, four legs are shown for connecting to the adapter 122. Each leg 178 extends in a substantially axial direction with respect to the needle hub. A distal end of the leg includes an inwardly projecting hook 180 for coupling with the annular wall 144. The hooks project radially inward and include a substantially flat inner face 182 and an inclined outer face 184.

The outer annular wall 144 of the adapter as shown in FIG. 9 includes an outwardly extending radial lip 146 forming the coupling mechanism. The lip 146 is formed by a substantially flat inner face 148 extending perpendicular to the longitudinal axis of the needle hub and an inclined outer face 150. The lip 146 is configured for coupling with the connecting legs 170 of the needle hub as shown in FIG. 7. The adapter 122 is connected to the delivery device as in the previous embodiment by the threaded connection. The needle hub 124 is then connected to the adapter by pressing the needle hub onto the adapter so that the projection penetrates the septum 160 and the inclined faces 150 of the lip 146 slide over the inclined faces 184 of the respective hook 180. The assembly can then be used for injecting the substance into the patient. After use, the needle hub can be separated from the adapter 122 and discarded. A new needle hub can then be attached to the adapter for a subsequent injection.

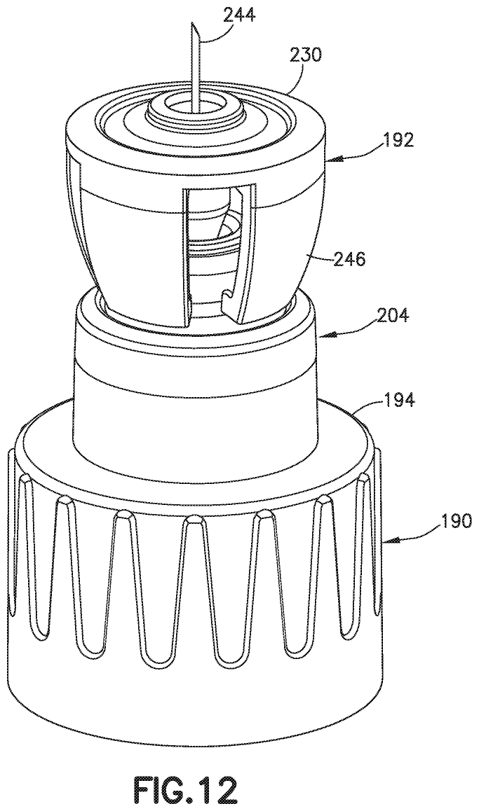

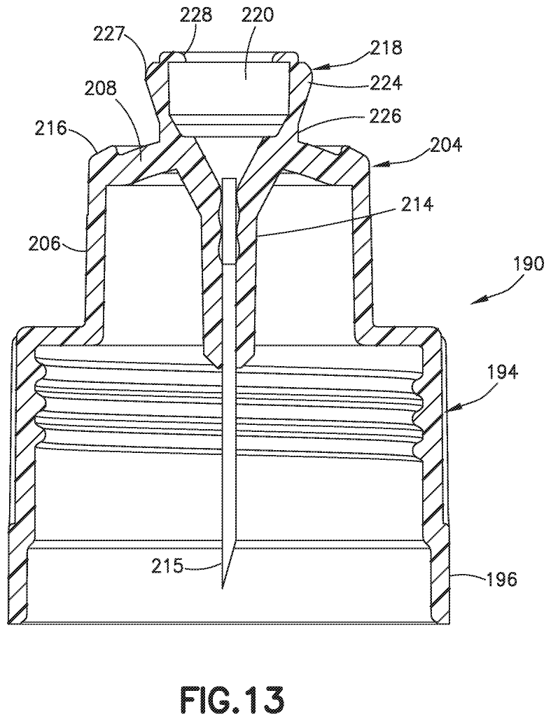

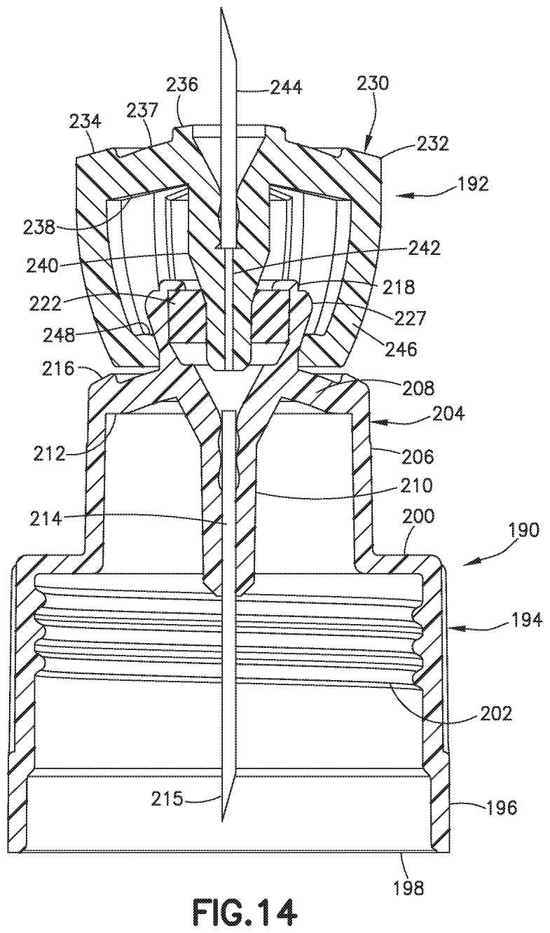

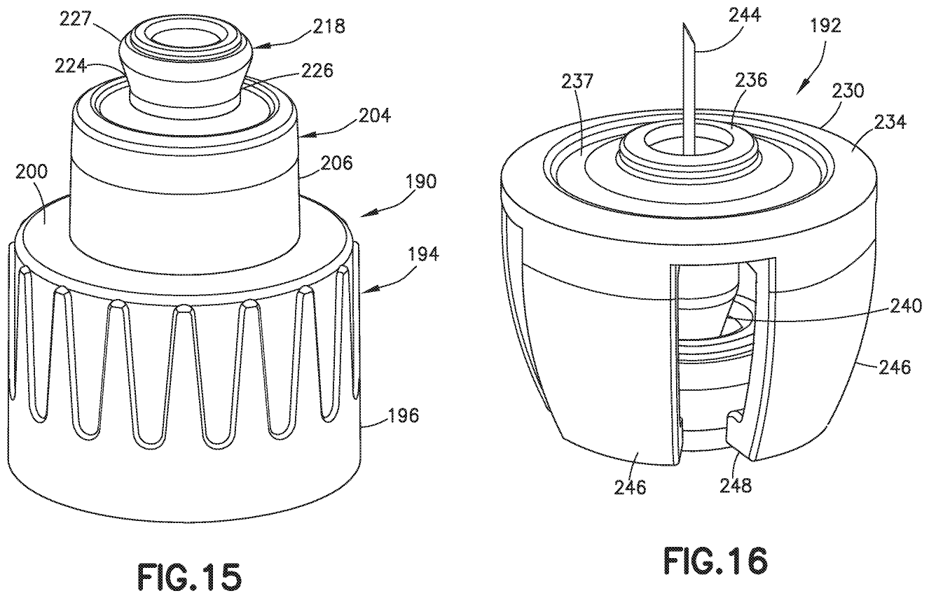

A further embodiment is shown in FIGS. 12-17 including an adapter 190 and a needle hub 192. The needle hub 192 is configured for connecting to a delivery device as in the previous embodiments. The adapter 190 includes a body 194 having a sidewall 196 with an open proximal end 198 as shown in FIGS. 13 and 14. A top wall 200 shown in FIG. 14 extends inwardly from the sidewall 196 at a distal end of the body 194. Internal threads 202 are provided for connecting to the delivery device.

An extension 204 projects from the top wall 200 in the distal direction. The extension 204 is formed by a sidewall 206 and an end wall 208. A projection 210 extends from an inner face 212 for forming the fluid connection between the needle hub 192 and the delivery device. The projection 210 is provided with a fluid passage 214. A cannula 215 extends from the projection 210 to provide fluid communication with the delivery device.

The end wall 208 has an outer distal face 216 with a coupling mechanism for coupling with the adapter 190. The coupling mechanism in the embodiment shown includes a collar 218 extending from the distal face 216. The collar 218 has an annular shape with an internal cavity 220 for receiving a septum 222 shown in FIG. 14. The collar 218 has a sidewall 224 with an outer surface forming an annular recessed area 226 facing radially outward. As shown in FIG. 13, sidewall 224 has an outer surface with a tapered profile so that the distal end of the sidewall 224 is wider than the proximal end to form the recessed area 226 next to the end wall 216 and an annular rib 227 at the distal end. An inwardly extending lip 228 extends inwardly toward the center axis to retain the septum 222 within the cavity 220 as shown in FIG. 14.

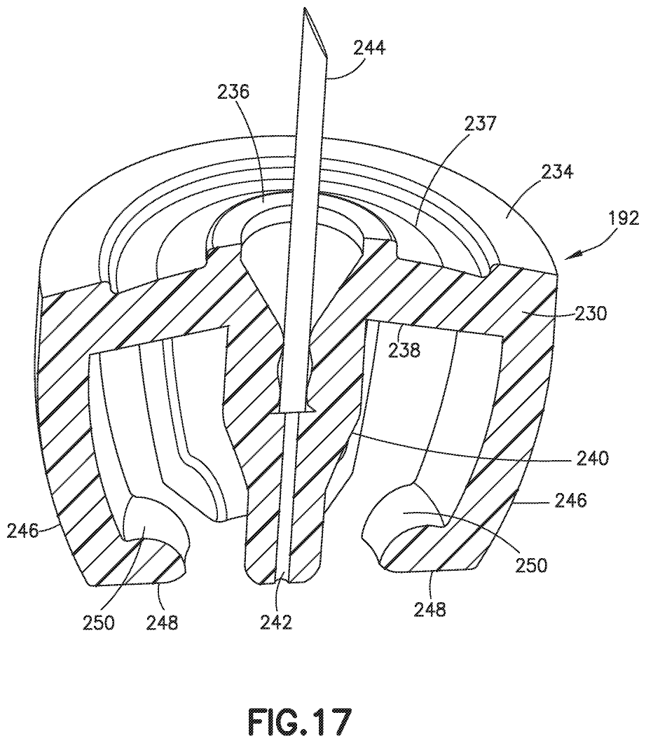

The needle hub 192 in the embodiment shown in FIGS. 16 and 17 has an end wall 230 with a distal face 232 forming a skin contact surface. The distal face 232, as in the previous embodiments, includes an outer annular surface 234 and inner annular surface 236 forming an annular recess 237 between the inner and outer surfaces. The dimensions of the surface 234, 236 and 237 can be the same configuration as in the previous embodiment.

End wall 230 has a proximal face 238 with a projection 240 extending from the proximal face. The projection 240 includes an internal passage 242 for supporting the needle 244 and providing fluid communication between the adapter 190 and the needle 244. A coupling mechanism is provided on the proximal face 238 for connecting to the adapter 190. In the embodiment shown in FIGS. 16 and 17, the coupling mechanism includes at least one and typically a plurality of legs 246 extending in the proximal direction around the perimeter of the end wall 230. The legs 246 extend from the proximal face 238 and include an inwardly extending hook 248 at the distal end of the legs 246. The hook 248 projects radially inward and has a shape and configuration for connecting to the coupling mechanism of the adapter. As shown in FIG. 14, the hook 248 has a dimension to complement the annular recessed area 226 for connecting the needle hub 192 to the adapter 190. As shown in FIG. 17, the hook 248 has a rounded top surface 250 for complementing the surface of the recessed area 226 of the collar 218.

In use, the adapter 190 is connected to the delivery device by the threaded connection for providing the fluid connection to the delivery device. The projection 210 is configured to penetrate a septum on the delivery device to provide a fluid connection. The septum 222 prevents leakage from the adapter 190. The needle hub 192 is then attached to the end of the adapter 190 as shown in FIG. 14 where the projection 240 penetrates the septum 222 to provide fluid connection between the delivery device and the needle 244. The device can then be used to inject the substance to the patient. After use, the used needle hub 192 can be removed and discarded.

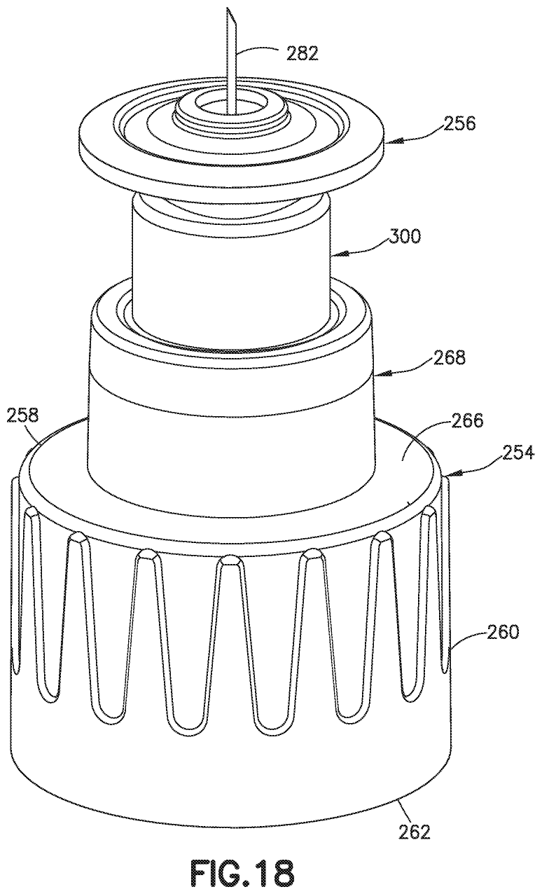

In the embodiment of FIGS. 18-21 the assembly includes an adapter 254 and a needle hub 256. The adapter 254 is configured for connecting to the delivery device as in the previous embodiment. The adapter includes a body 258 with a depending side wall 260 having an open proximal end 262 as shown in FIG. 18. Internal threads provide a threaded connection to the delivery device as in the previous embodiment. A top wall 266 extends inwardly from the distal end of the wall 260. As in the previous embodiments, a projection having a cannula extends from the inner face of the top wall 266 for forming the fluid communication with the delivery device.

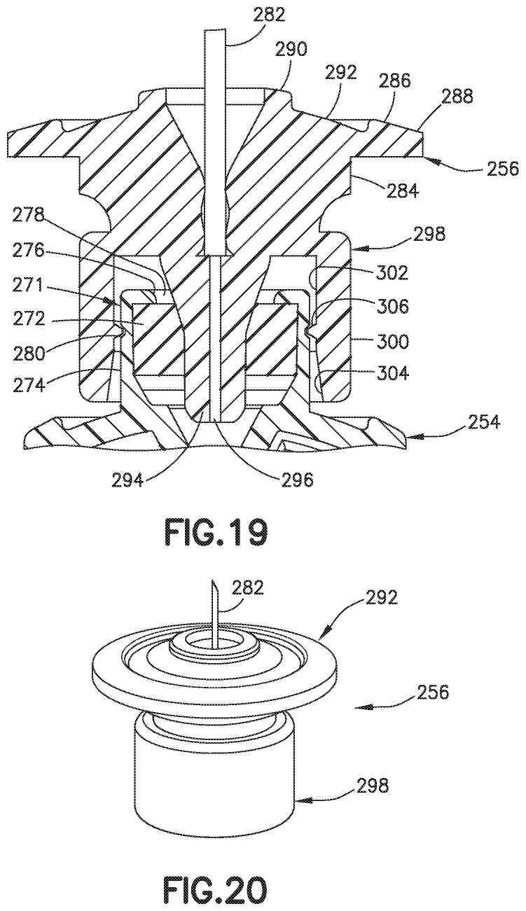

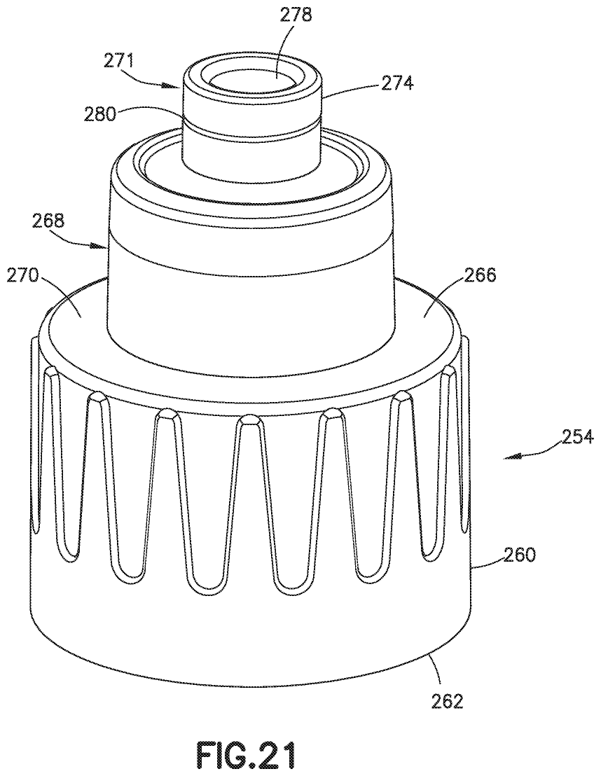

An extension 268 projects from the distal face 270 of the top wall 266. A collar 271 extends from the distal end of the extension 268 to form an internal cavity for receiving a septum 272. The collar 271 has a side wall 274 having a substantially cylindrical configuration and an inwardly extending lip 276 forming a distal opening 278. In the embodiment shown, the outer surface of the side wall 274 includes an annular groove 280 facing radially outward shown in FIGS. 19 and 21.

The needle hub 256 is configured for supporting a needle 282 and for connecting to the adapter 254. The needle hub 256 includes a body 284 having a radial flange having a distal face 286 forming a skin contact surface shown in FIG. 19. The distal face 286 includes an outer annular surface 288, an inner annular surface 290 and an annular recess 292. A proximal end of the body 284 includes a projection 294 having an axial fluid passage 296 to form a fluid connection with the adapter and delivery device. As shown in FIG. 19, the needle 282 is received in the passage 296 and projects from the distal face of the needle hub 256.

A collar 298 extends from the proximal end of the body 284 of the needle hub 256 and is formed by an annular wall 300. The wall 300 has an inner surface 302 with a tapered end portion 304. A projection shown as an annular rib 306 projects radially inward from the inner surface 302 of the wall 300 for coupling with the annular groove 280 of the extension 268. In an alternative embodiment, the groove can be formed on the inner face of the collar 298 and the annular rib can be formed on the outer surface of the wall 274. During use, the adapter 254 is connected to the delivery device for providing fluid connection. The needle hub 256 is then attached to the adapter as shown in FIG. 19 where the rib 306 is received in the annular groove 280.

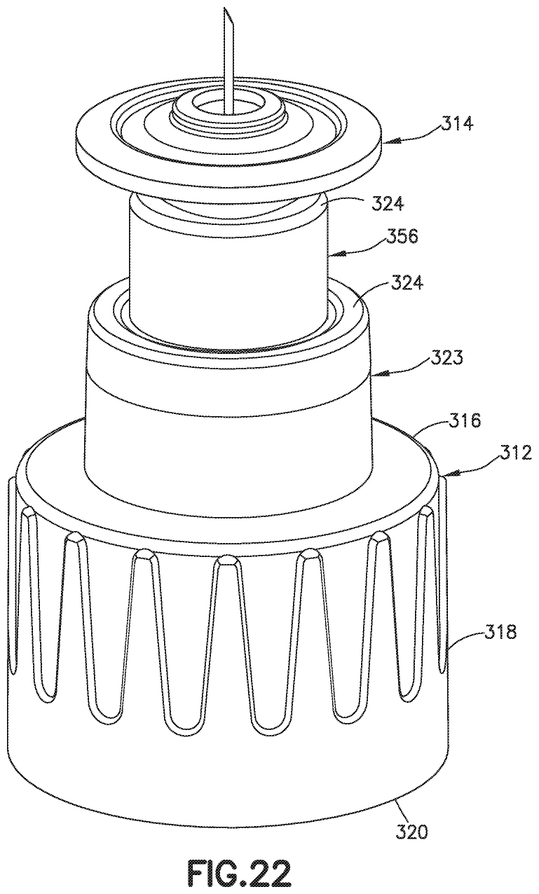

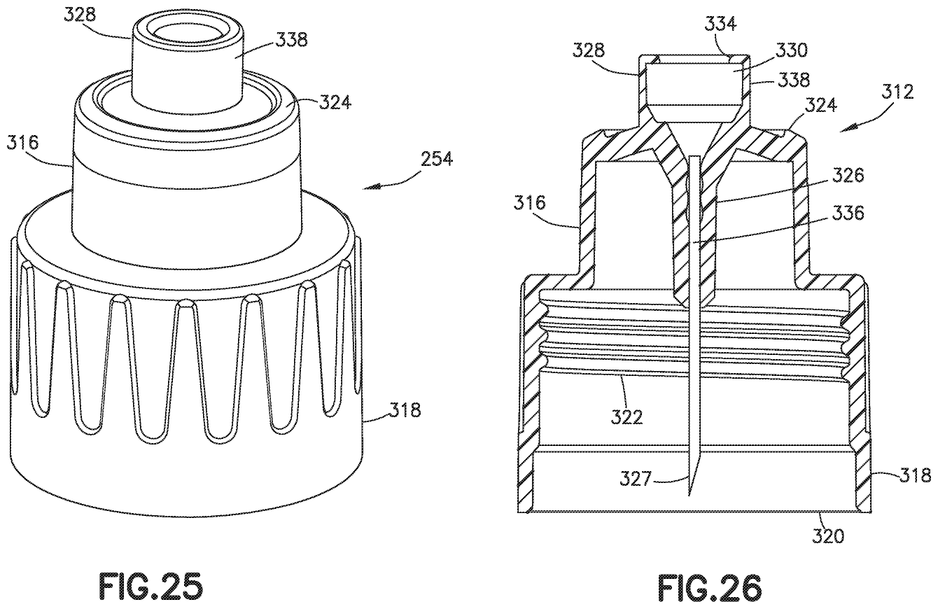

Referring to FIGS. 22-26, another embodiment is illustrated that is similar to the embodiment of FIGS. 18-21. The adapter 312 is configured for connecting to the delivery device and for connecting to the needle hub 314. The adapter 312 as in the previous embodiment includes a body 316 with a sidewall 318 extending to an open distal end 320. Internal threads 322 are formed on the inner surface of the sidewall 318 for connecting to the delivery device. An extension 323 having an end wall 324 is formed at the distal end of the body 316. A projection 326 having a fluid passage 336 extends from an inner surface of the end wall 324 to form the fluid connection with the delivery device. A needle 327 extends from the projection 326.

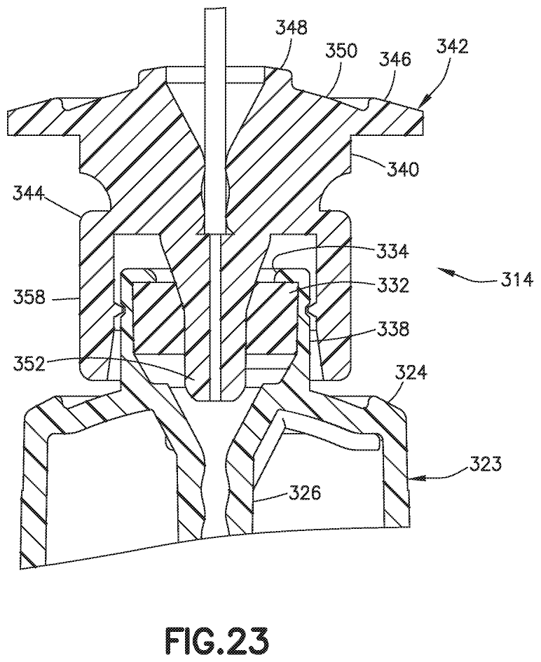

A collar 328 extends in the distal direction from the end wall 324 to form a cavity 330 shown in FIG. 26 to support a septum 332 shown in FIG. 23. An inwardly extending lip 334 retains the septum to within a cavity 330. In the embodiment shown in FIG. 26, the collar 328 as a cylindrical sidewall 338 with a substantially smooth or slightly tapered outer surface.

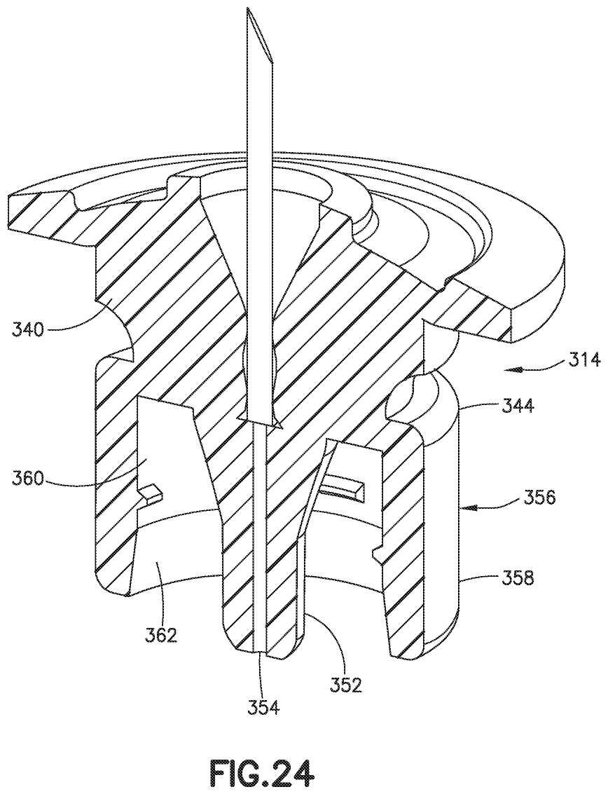

The needle hub 314 is similar to the previous embodiment by including a body 340 having a radially extending flange with a distal face 342 and a proximal end 344 shown in FIG. 23 and FIG. 24. The distal face 342 as in the previous embodiment as a distal outer annular surface 346, a distal inner annular surface 348 and an annular recess 350. The proximal end 344 includes a projection 352 having an axial passage 354 to connect with the adapter 312. The collar 356 extends from the proximal end 344 and is formed by an annular wall 358 having a substantially smooth inner surface 360. A distal end of the wall 358 has a tapered portion to assist connecting the needle hub 314 to the adapter 312. In the embodiment shown, the inner surface 360 of the wall 358 has a dimension to form a friction fit with the outer surface of the collar 328. In use, the adapter 312 is connected to the delivery device and the needle hub is attached to the adapter as shown in FIG. 23.

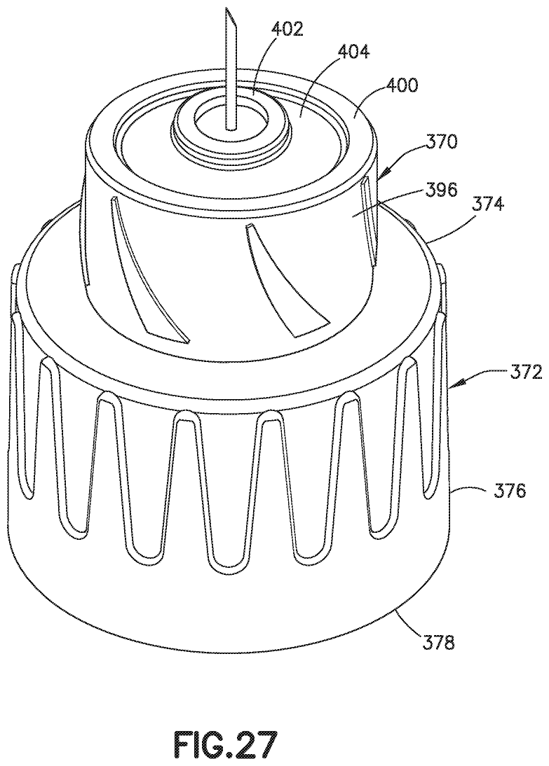

A further embodiment shown in FIGS. 27-30 includes an adapter 370 and a needle hub 372. The adapter 370 as in the previous embodiments is configured for connecting to a delivery device. The adapter 370 has a body 374 and a sidewall 376 forming an open proximal end 378. Internal threads are provided on the sidewall 376 for connecting to the delivery device.

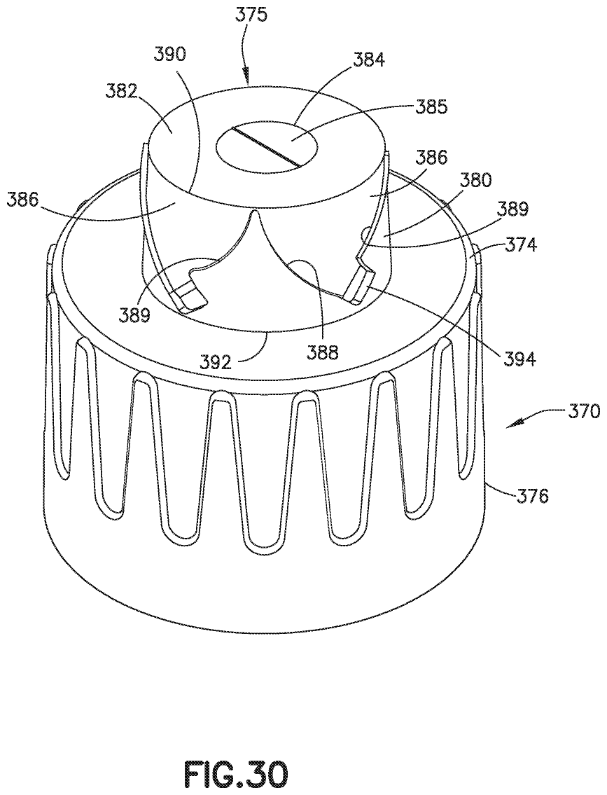

An extension 375 extending from the body 374 of the embodiment shown in FIG. 30 has a generally cylindrical configuration with an outer surface 380 and an axially extending end wall 382 having an opening 384 for providing fluid communication between the adapter 370 and needle hub 372. As in the previous embodiment, a septum 385 is provided in the opening 384. The adapter 370 has a projection and a needle extending from the proximal end of the body 374 as in the previous embodiment for connecting with the delivery device.

As shown in FIG. 30 the outer surface 380 of the body 375 is formed with a plurality of recesses forming internal threads 386 of a threaded connection with the needle hub. The recesses 386 have generally curved edge 388 and a converging curved edge 389 extending from a distal end 390 of the body 375 to a proximal end 392 of the body. A recess 394 is formed at the end of the internal threads 386.

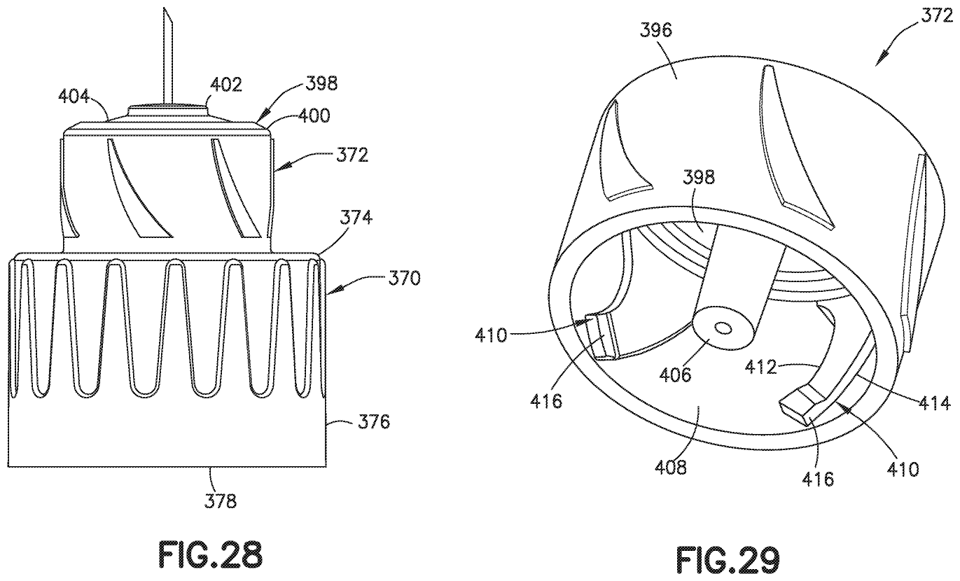

The needle hub 372 as shown in FIG. 29 has a sidewall 396 and an end wall 398. The outer surface of the end wall 398 forms a skin contact surface defined by an outer annular surface 400, an inner annular surface 402, and an annular recess 404 shown in FIG. 27 and FIG. 28. A projection 406 extends from an inner surface of the end wall 398 as shown in FIG. 29 to form a fluid connection with the adapter 370. The sidewall 396 has an inner surface 408 with a plurality of raised portions forming threads 410 complementing the internal threads 386. The threads 410 have a concave leading surface 412 and a convex trailing surface 414. A projection 416 projects inwardly at the proximal end of the threads 410. The projection 416 to has a shape and dimension for complementing the recess 394 of the threads 386. The outer surface of the side wall in the embodiment shown has spiral shaped ridges to assist the user in the gripping and rotating of the needle hub 372.

The threaded coupling mechanism formed by the threads 410 and 386 have a pitch for quick and easy attachment and removal of the needle hub to the adapter. The pitch as shown in FIG. 29 and FIG. 30 is less than the axial length of the body 374 so that the needle hub can be attached by less than one full turn of the needle hub relative to the adapter. In the embodiment shown, the pitch of the threads 386 and 410 is about one fourth the circumference of the outer face of the body. The curved surfaces enable quick attachment and removal while the projection on the thread 416 follow the curvature of the threads 386 and snap into the recess 394 for provide a tactile or audible indication that the needle hub is properly attached and connected to the adapter.

The threaded coupling mechanism allows the needle hub to be connected to and removed from the adapter by a short twist of the needle hub relative to the adapter so that the needle hub is easily connected to the adapter and easily removed from the adapter. For some patients, gripping and twisting the small needle hub due the small dimensions and the tight fit can be difficult. The threaded coupling also allows replacement of the needle hub with a new needle hub for a subsequent injection.

In the embodiment shown, the coupling mechanism has complementing threads although other coupling mechanisms can be used. In another embodiment, the collar of the adapter can include a detent projecting inward from the side wall to mate with a spiral groove formed on the inner surface of the needle hub. In other embodiments, the inner surface of the needle hub can have a detent or spiral thread projecting radially inward toward the center axis of the inner shield to mate with a spiral groove formed on the adapter. In still further embodiments, the coupling mechanism can be a bayonet type connection where the post or the inner shield include an L-shaped groove that mates with a detent on the other of the post or inner shield.

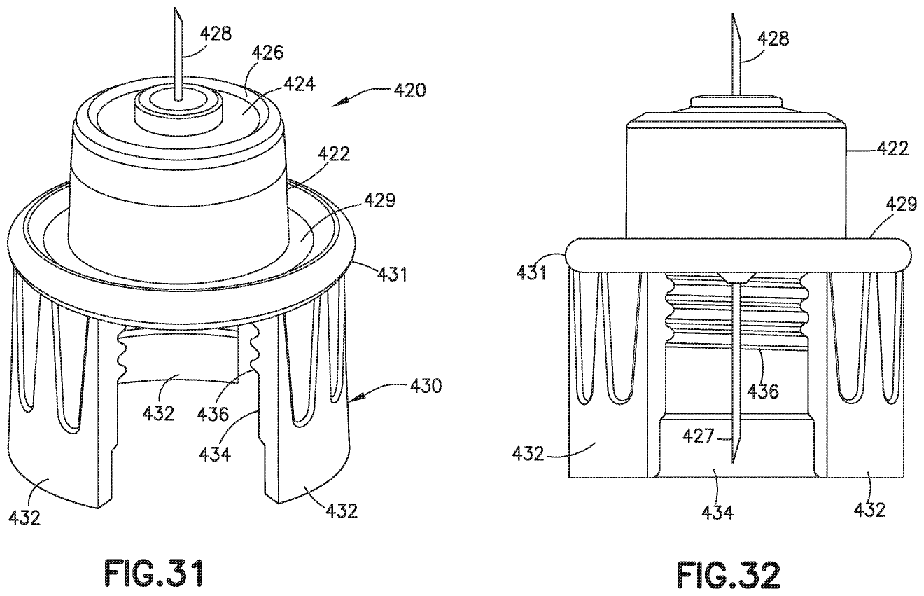

FIGS. 31-38 illustrate other embodiments of the assembly where the assembly is a one-piece integrally formed unit. The needle hub 420 shown in FIGS. 31 and 32 has a body 422 with an end wall 424 forming a distal face 426 forming the skin contact surface and having a needle 428 extending from the distal face 426 for injecting the substance to the patient. A projection having a needle 427 extends from the inner surface of the end wall 424 for connecting to the delivery device.

A shoulder 429 extends radially outward from the body 422 to define a peripheral edge 431. A depending side wall 430 extends from the peripheral edge 431 of the shoulder 429 for connecting to the delivery device. In the embodiment shown, the side wall 430 is formed as spaced apart sections 432 having a concave inner surface 434 with internal threads 436 and a convex outer surface 438. In one embodiment, the three sections 432 are provided for coupling to the delivery device. The sections are sufficiently flexible so that the needle hub is attached to the delivery device by pressing onto the threaded connection of the delivery device. The needle hub can then be tightened by twisting the needle hub onto the delivery device to form a fluid connection with the delivery device.

FIGS. 33 and 34 show another embodiment where the needle hub 440 has a body 442 with an end wall forming a distal surface and a needle 444 extending from the distal surface. A shoulder 446 extends radially outward to a peripheral edge 448. A depending side wall 450 extends from the peripheral edge for connecting with the delivery device.

As shown in FIG. 34, the depending side wall 450 has a plurality of cut out sections 452 forming flexible tabs that can deflect outward when attached to the delivery device. Internal threads 454 on the inner surface of the side wall 450 forms the threaded connection with the delivery device. In the embodiment shown, the cut out sections 452 are oriented outwardly from the side wall 450 as shown in FIG. 34.

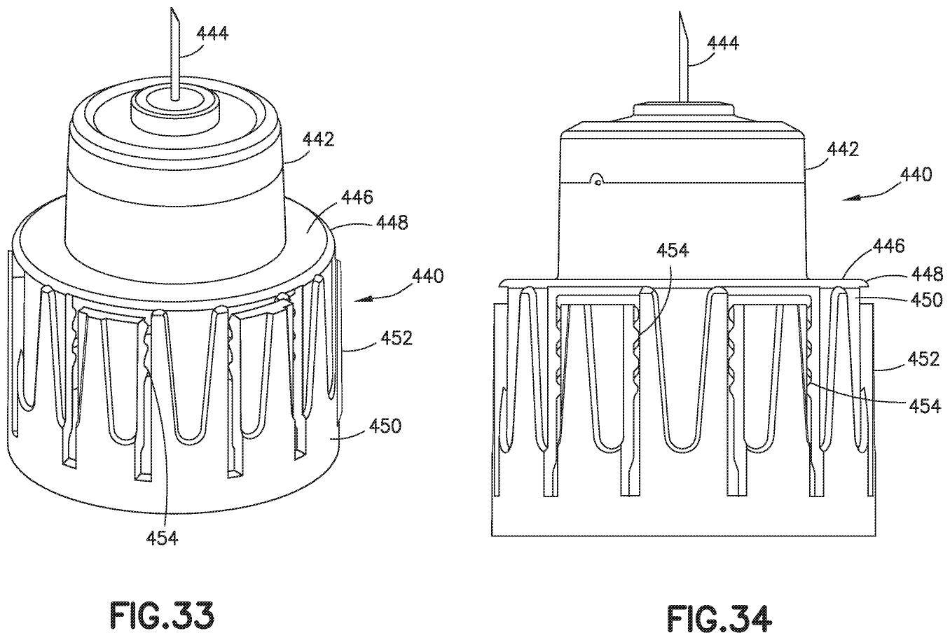

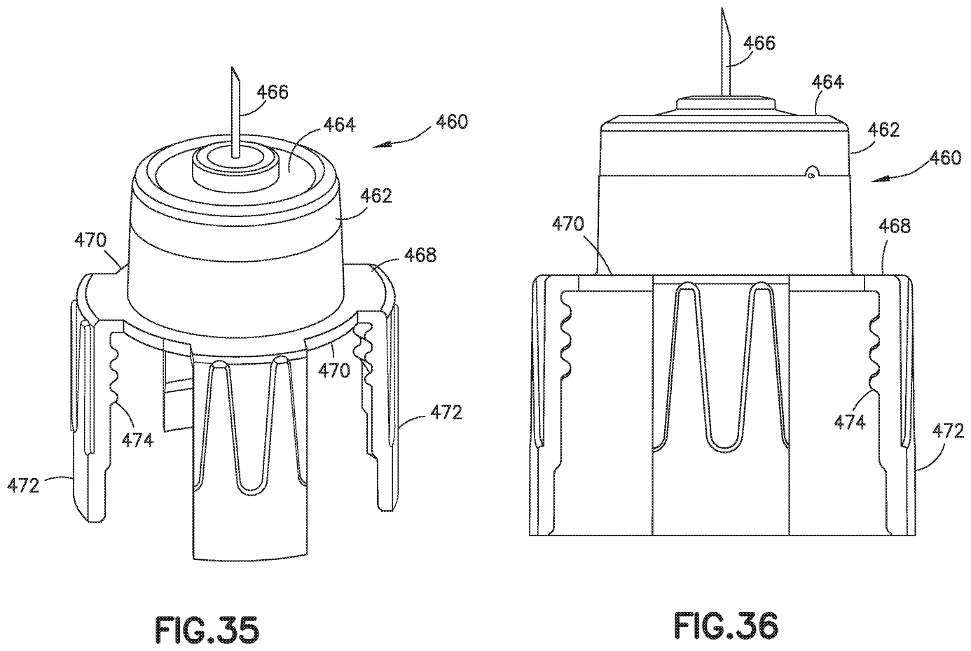

FIGS. 35 and 36 show and embodiment of the needle hub 460 similar to the needle hub of FIGS. 31 and 32. The needle hub 460 has a body 462 with an end wall 464 having a needle 466 extending from the end wall for injecting the substance to the patient. A shoulder 468 extends radially outward from the body 462 to a peripheral edge 470. In the embodiment shown, four spaced apart legs 472 depend from the shoulder at the peripheral edge 470 to form a side wall. The inner concave surface of the legs 472 have internal threads 474 for coupling with the delivery device. The legs are sufficiently flexible so that the needle hub can be pressed onto the end of the delivery device and twisted to tighten and form a fluid connection with the delivery device.

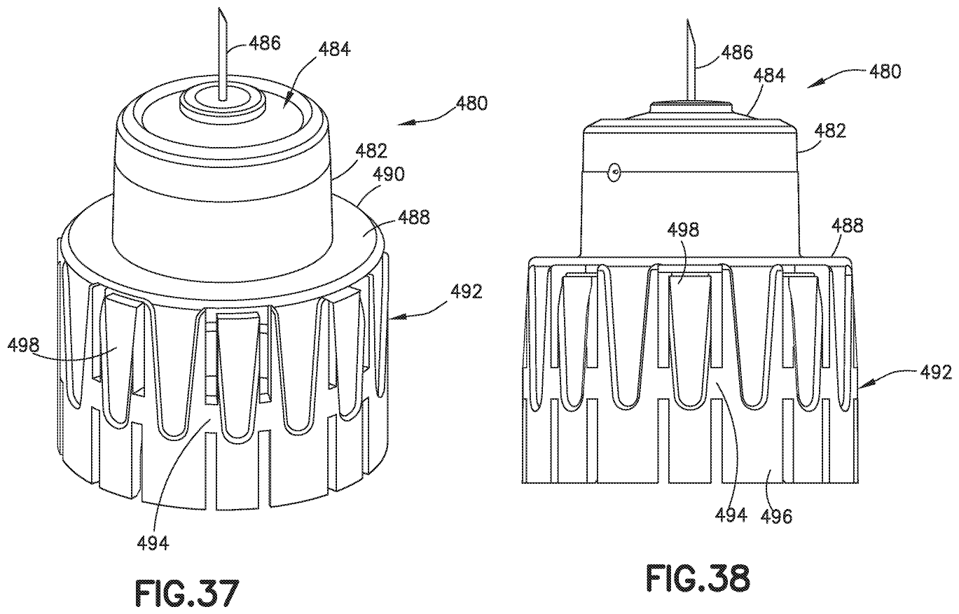

FIGS. 37 and 38 show another embodiment of the needle hub 480 having a body 482 with an end wall 484 and a needle 486 extending from the end wall 484. A shoulder 488 extends radially outward from a proximal end of the body 482 to a peripheral edge 490. A depending side wall 492 extends from the peripheral edge 490 for connecting the delivery device. The side wall 492 has internal threads for connecting to the delivery and forming the fluid connection.

As shown in FIG. 38, the side wall 492 has a mid-section 494 forming a continuous wall extending around the hub 480. A plurality of depending legs 496 extend from the proximal edge of the side wall and a plurality of depending legs 498 extend toward the distal end of the side wall toward the shoulder 488. The legs 498 include internal threads for connecting the needle hub to the delivery device. Each of the legs 496 and 498 are sufficiently flexible to engage the threads of the delivery device while enabling quick and easy separation.

The foregoing embodiments and advantages are merely exemplary and are not to be construed as limiting the scope of the present invention. The description of an exemplary embodiment of the present invention is intended to be illustrative, and not to limit the scope of the present invention. Various modifications, alternatives, and variations will be apparent to those of ordinary skill in the art, and are intended to fall within the scope of the invention. It is particularly noted that the features of different embodiments and claims may be combined with each other as long as they do not contradict each other. Accordingly, all such modifications are intended to be included within the scope of this invention as defined in the appended claims and their equivalents.

1. A pen needle assembly comprising:
an adapter having a side wall with a proximal end configured for coupling to a pen needle delivery device, a distal end, and an end wall at said distal end, and said end wall having a fluid passage; and
a needle hub having a distal end with a distal face, a needle extending from said distal face for delivering a substance from the pen needle delivery device, and a proximal end removably coupled to said adapter to provide fluid communication between the needle and the delivery device.

2. The pen needle assembly of claim 1, wherein said side wall of said adapter includes internal threads for mating with a threaded outlet end of the pen needle delivery device, and said end wall of said adapter includes a post and needle extending toward said proximal end for coupling with the pen needle delivery device.

3. The pen needle assembly of claim 1, wherein said adapter includes a septum positioned in said fluid passage, and said needle hub includes a post for penetrating said septum and providing fluid communication between said fluid passage and said needle.

4. The pen needle assembly of claim 3, wherein said end wall of said adapter has a distal face with a recess surrounding said fluid passage, and where said septum is received in said recess to close said fluid pathway.

5. The pen needle assembly of claim 1, wherein said distal face has a convex surface and a dimension to define the skin contact surface surrounding said needle.

6. The pen needle assembly of claim 5, wherein said distal face of said needle hub has an annular recess.

7. The pen needle assembly of claim 1 wherein said adapter has at least one coupling member for coupling with said needle hub, and where said needle hub is separable from said adapter.

8. The pen needle assembly of claim 7, wherein said at least one coupling member on said adapter comprises a plurality of hook members projecting inwardly with respect to a center axis of said adapter, and where said needle hub has a coupling member for coupling with said hook members.

9. The pen needle assembly of claim 8, wherein said coupling member on said needle hub comprises an outwardly open annular recess.

10. The pen needle assembly of claim 7, wherein said at least one coupling member on said needle hub comprises at least one hook member projecting radially inward with respect to a longitudinal axis of said needle hub, and where said coupling member of said adapter extends outwardly for coupling with said at least one hook member.

11. The pen needle assembly of claim 7, wherein said at least one coupling member on said needle hub comprises a plurality of axially extending legs, each of said legs having a projection extending radially inward, and said coupling member of said adapter comprises an open annular recess configured for mating with said projection on said plurality of legs.

12. The pen needle assembly of claim 7, wherein said at least one coupling member on said needle hub comprises an axially depending collar having an inner surface with an inwardly extending projection, and where said coupling member of said adapter has an outer surface for mating with said projection by a press fit.

13. The pen needle assembly of claim 7, wherein said needle hub has an axially depending side wall with an inner surface, and where said at least one coupling member on said needle hub comprises internal threads, and said coupling member on said adapter comprises a threaded coupling for mating with said internal threads of said needle hub.

14. The pen needle assembly of claim 13, wherein said internal threads on said needle hub and said threaded coupling on said adapter have a pitch greater than the longitudinal dimension of said internal threads.

15. A pen needle assembly comprising:
an adapter having a side wall with an open proximal end and an end wall at the distal end, said distal end having a coupling member; and
a needle hub having a proximal end, a distal face, and a needle extending from said distal face, said needle hub configured for coupling with said coupling member of said adapter, and said distal face adapted for forming a fluid communication of said needle with said adapter.

16. The pen needle assembly of claim 15 wherein said outer surface of said post includes a spiral thread, and said threaded coupling mechanism of said inner shield comprises a spiral thread on an inner surface of said inner shield complementing said spiral thread on said post.

17. A pen needle assembly comprising:
a body having a distal end, a proximal end, and an end wall with an axial face at said distal end, and a needle extending from said end wall; and
a collar extending from said proximal end of said body, said collar having a substantially cylindrical configuration with an outer surface and an inner surface, said inner surface having internal threads for coupling to a threaded end of the pen needle delivery device, said collar having a plurality of flexible legs for coupling with the threaded end of the pen needle delivery device.

18. The pen needle assembly of claim 17, wherein said collar comprises said plurality of flexible legs, said plurality of flexible legs extending in an axial direction with respect to said proximal end of said body and spaced apart a distance to define a gap between adjacent legs.

19. The pen needle assembly of claim 17, wherein said collar has a continuous ring forming a bottom portion, and where said plurality of legs extending from said bottom portion in a direction toward said proximal end of said body.

20. The pen needle assembly of claim 17, wherein said plurality of legs include a first set of flexible legs extending in an axial direction toward said proximal end of said body, and a second set of flexible legs extending away from said proximal end of said body.

21. A method of assembling a pen needle assembly on a pen needle delivery device comprising the steps of:
coupling the pen needle adapter to the pen needle delivery device, where said pen needle adapter has a body and a collar for coupling to the pen needle delivery device, said body having at least one coupling member; and
coupling a pen needle hub to the adapter, said adapter having a distal face defining a skin contact surface and a needle projecting from said skinned contact face, and where said needle is in fluid communication with said adapter.
